(19) United States
(12) Patent Application Publication
(10) Pub. No.: US 2007/0092913 A1
(43) Pub. Date: Apr. 26, 2007

(54) Methods for identifying agents that modulate GPR105 activity

(75) Inventors: John Lamb, Shoreline, WA (US); Joseph Mancini, Kirkland (CA); Brian Kennedy, Kirkland (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignees: Rosetta Inpharmatics LLC, Seattle, WA (US); Merck and Co., Inc., Rahway, NJ (US)

(21) Appl. No.: 11/584,424

(22) Filed: Oct. 19, 2006

Related U.S. Application Data
(60) Provisional application No. 60/730,595, filed on Oct. 26, 2005.

(51) Int. Cl. G01N 33/53; C07H 21/04; C12P 21/06; C07K 14/705

(52) U.S. Cl. 435/7.1; 435/69.1; 435/320.1; 435/325; 530/350; 536/23.5

(57) ABSTRACT

In one aspect, the present invention provides methods for determining whether a chemical agent modulates the biological activity of a GPR105 protein. The methods of this aspect of the invention include the steps of: (a) contacting a living cell, in vitro, with a chemical agent, wherein the living cell expresses a GPR105 protein having a biological activity; (b) determining whether the chemical agent modulates the biological activity of the GPR105 protein in the living cell; and (c) validating, in vivo, the modulating effect of the chemical agent on the biological activity of the GPR105 protein.

### CROSS-REFERENCE TO RELATED APPLICATION

This application claims the benefit of U.S. Provisional Application No. 60/730,595, filed Oct. 26, 2005.

### FIELD OF THE INVENTION

The present invention relates to methods for screening for new drug molecules.

### BACKGROUND

Metabolic Syndrome is a disorder that includes obesity, dyslipidaemia, and hyperglycemia. Metabolic Syndrome has increased to epidemic proportions worldwide. The pathophysiology of this syndrome is attributed to central distributed obesity, decreased high density lipoprotein, elevated triglycerides, elevated blood pressure and hyperglycemia. People suffering from Metabolic Syndrome are at increased risk of type II diabetes, coronary heart disease, and other diseases related to plaque accumulation in artery walls (e.g., stroke and peripheral vascular disease). In two prospective European studies, Metabolic Syndrome was a predictor of increased cardiovascular disease and mortality. (Isomaa et al., “Cardiovascular Morbidity and Mortality Associated With the Metabolic Syndrome,” Diabetes Care 24:683-689, 2001; Lakka et al., “The Metabolic Syndrome and Total and Cardiovascular Disease Mortality in Middle Aged Men,” JAMA 288:2709-2716, 2002.)

The most significant underlying cause of Metabolic Syndrome is obesity. The genetic factors that also contribute to Metabolic Syndrome are not yet understood. Consequently, there is a need to identify genes that contribute to the development of Metabolic Syndrome. There is also a need for methods that permit the identification of chemical agents that modulate the activity of these genes or modulate the activity of the products (e.g., proteins) encoded by these genes. Such chemical agents may be useful, for example, as drugs to prevent Metabolic Syndrome or to ameliorate at least one symptom of Metabolic Syndrome.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with the foregoing, the present inventors have discovered that expression of GPR105 protein is correlated with weight gain and development of type II diabetes. Further, the present inventors have demonstrated that antisense inhibition of GPR105 expression in mice reduces the rate at which the mice gain weight in response to a high fat diet. The mice also have lower levels of insulin, suggesting a decreased level of insulin resistance in these mice. Accordingly, GPR105 is a target for drugs that prevent diabetes, obesity or Metabolic Syndrome, or that ameliorate at least one symptom of Metabolic Syndrome.

Thus, in one aspect, the present invention provides methods for determining whether a chemical agent modulates the biological activity of a GPR105 protein. The methods of this aspect of the invention include the steps of: (a) contacting a living cell, in vitro, with a chemical agent, wherein the living cell expresses a GPR105 protein having a biological activity; (b) determining whether the chemical agent modulates the biological activity of the GPR105 protein in the living cell; and (c) validating, in vivo, the modulating effect of the chemical agent on the biological activity of the GPR105 protein.

In another aspect, the present invention provides methods for determining the effect of a chemical agent on GPR105 activity. The methods of this aspect of the invention each include the steps of: (a) observing a change in a GPR105-mediated response in a living cell, in vitro, in response to a chemical agent; and (b) confirming that the observed change in the GPR105-mediated response occurs in vivo.

The foregoing methods of the present invention are useful, for example, for identifying chemical agents that modulate the biological activity of a GPR105 protein in a living cell. These chemical agents are useful, for example, as drugs to prevent obesity or diabetes, or to ameliorate at least one symptom of Metabolic Syndrome, or to further characterize the mechanism of action of a GPR105 protein in a living cell. The foregoing methods of the present invention can be used, for example, as an initial screen to identify chemical compounds that can be further screened and analyzed to identify compounds that prevent obesity or type II diabetes or that ameliorate at least one symptom of Metabolic Syndrome.

### DETAILED DESCRIPTION

The present invention provides methods for determining whether a chemical agent modulates the biological activity of a GPR105 protein. The methods comprise the steps of: (a) contacting a living cell, in vitro, with a chemical agent, wherein the living cell expresses a GPR105 protein having a biological activity; (b) determining whether the chemical agent modulates the biological activity of the GPR105 protein in the living cell; and (c) validating, in vivo, the modulating effect of the chemical agent on the biological activity of the GPR105 protein.

As used herein, the term “chemical agent” encompasses any chemical molecule, or chemical element, or a combination of chemical molecules and/or chemical elements. For example, the term “chemical agent” encompasses proteins (comprising at least 100 covalently linked amino acid units) and peptides (comprising from 2 to 99 covalently linked amino acid units). Again, by way of example, the term “chemical agent” encompasses molecules that mimic the ligands for a GPR105 protein.

As used herein, the term “GPR105 protein” refers to a type of G-protein coupled receptor. The natural ligand for GPR105 protein is not known, but UDP-hexoses (e.g., UDP-glucose) binds the receptor with high affinities (100-500 nM). UDP-glucose is an activated form of glucose used for glycogen synthesis. The biological function of GPR105 is not known, but it may have a role in cellular chemotaxis and inflammation. Human body atlas data shows that GPR105 is predominantly expressed in the intestines and subcutaneous white adipose tissue. Mouse data shows highest expression of GPR105 in spleen and pancreas, with only average expression in brain.

Some GPR105 proteins useful in the practice of the present invention are at least 79% identical (e.g., at least 80% identical, or at least 90% identical, or at least 95% identical, or at least 99% identical) to the human GPR105 protein having the amino acid sequence set forth in SEQ ID NO:1, and encoded by the transcript having GenBank accession number NM_{—}014879.

The term “percent identity” or “percent identical,” when used in connection with amino acid sequence relatedness between GPR105 proteins, is defined as the percentage of amino acid residues in a first GPR105 protein sequence that are identical with a second GPR105 protein sequence (such as the GPR105 amino acid sequence of SEQ ID NO: 1), after aligning the first and second GPR105 sequences to achieve the maximum percent identity. For example, percentage identity between two protein sequences can be determined by pairwise comparison of the two sequences using the b12seq interface at the Web site of the National Center for Biotechnology Information (NCBI), U.S. National Library of Medicine, 8600 Rockville Pike, Bethesda, Md. 20894, U.S.A. The b12seq interface permits sequence alignment using the BLAST tool described by A. Tatiana et al., “Blast 2 Sequences—A New Tool for Comparing Protein and Nucleotide Sequences,” FEMS Microbiol Lett. 174:247-250, 1999. The following alignment parameters are used: Matrix=BLOSUM62; Gap open penalty=11; Gap extension penalty=1; Gap x_dropff=50; Expect=10.0; Word size=3; and Filter=off.

As used herein, the term “biological activity” refers to an effect of a GPR105 protein on a biological process in a living cell, living tissue, living organ and/or living organism. Examples of biological processes include biochemical pathways, concentration of one or more chemical compounds within a living cell, physiological processes that contribute to the internal homeostasis of a living organism, developmental processes that contribute to the normal physical development of a living organism, and acute or chronic diseases.

Modulation of the biological activity of a GPR105 protein encompasses any change in a biological activity of a GPR105 protein. For example, the change can be a decrease in a biological activity of a GPR105 protein (e.g., complete, or substantially complete, inhibition of a biological activity of a GPR105 protein). Again by way of example, the change can be a reduction in the rate of a biological activity of a GPR105 protein. Again by way of example, the change can be an increase in the activity of a GPR105 protein.

In the practice of the invention, a living cell, more typically a population of living cells, such as a liquid culture of living cells, is/are contacted with a chemical agent. It is then determined whether the chemical agent modulates the biological activity of a GPR105 protein in the living cell. By way of example, modulation of the biological activity of GPR105 protein in a living cell can be identified using the method disclosed by P. Kunapuli et al., Analytical Biochemistry 314:16-29, 2003, which publication is incorporated herein by reference. In brief, a vector that includes a nucleic acid molecule encoding a GPR105 protein is stably introduced into cells, such as HEK cells or CHO cells, and the encoded GPR105 protein is expressed in the cells. Additionally, a nucleic acid molecule encoding β-lactamase is stably introduced into the cells expressing the GPR105 protein. Thereafter, the cells are contacted with a candidate agonist, or candidate antagonist, of GPR105 and the effect of the candidate agonist, or candidate antagonist, on the expression of β-lactamase in the cells is measured (e.g., to determine whether the candidate agonist or antagonist causes an increase or decrease of expression of β-lactamase in the cells). Examples of methods for measuring β-lactamase activity in living cells are set forth, for example, in J. K. Chambers et al., “AG Protein-Coupled Receptor for UDP-Glucose,” J. Biol. Chem. 275(15):10767-10771, 2000; and P. Kunapuli et al., supra. Alternatively, the effect of the candidate agonist, or candidate antagonist, on GPR105 activity can be assessed by measuring changes in intracellular calcium levels in response to the action of the candidate agonist or candidate antagonist on GPR105.

By way of example, the cDNA molecule disclosed in SEQ ID NO:2 encodes a chimpanzee GPR105 protein (SEQ ID NO:3) that is useful in the practice of the present invention. As described more fully in Example 2, the nucleic acid molecule shown in SEQ ID NO:2 was modified and cloned into a pcDEF3 expression vector and co-transfected with a vector containing Gqi5 into HEK-293 cells so that the transfected cells expressed GPR105 protein (SEQ ID NO:3). The cells were maintained for several days and then plated into a 384 well format and challenged with various concentrations of UDP-glucose. Measurement of Ca²⁺stimulation in these HEK cells was performed using FLIPR (Molecular Devices, CA, USA) as previously described (K. Freeman et al., “Cloning, Pharmacology, and Tissue Distribution of G-Protein-Coupled Receptor GPR105 (KIAA0001) Rodent Orthologs,” Genomics 78:124-128, 2001).

An assay for assessing the effect of a chemical agent on a biological activity of a GPR105 protein typically includes at least one experimental treatment wherein a living cell (or population of living cells), expressing a GPR105 protein, is/are contacted with a chemical agent; and a control treatment wherein the same type of cell(s) expressing the GPR105 protein (e.g., an aliquot of the same preparation of cells used in the experimental treatment) is/are treated identically to the cell(s) used in the experimental treatment, except that the cell(s) used in the control treatment is/are not contacted with the chemical agent. Comparison of the GPR105 biological activity in the experimental treatment(s) with the GPR105 biological activity in the control treatment(s) permits determination of whether the chemical agent modulates GPR105 biological activity. For example, a level of GPR105 biological activity that is significantly lower in the experimental treatment(s) compared to the control treatment(s) indicates that the chemical agent inhibits GPR105 biological activity. Again by way of example, a level of GPR105 biological activity that is significantly higher in the experimental treatment(s) compared to the control treatment(s) indicates that the chemical agent stimulates GPR105 biological activity.

Numerous assays (e.g., hundreds or thousands of assays) for assessing the effect of a chemical agent on GPR105 biological activity can be automated and conducted simultaneously. For example, the assays can be automated in a high-throughput sequence format as described, for example, by O. Kornienko et al., J. Biomol. Screen 9(3):186-195, 2004.

In the practice of the claimed methods, the modulating effect of a chemical agent on GPR105 biological activity is validated in vivo. The validation step shows that a chemical agent that modulates the biological activity of GPR105, in vitro, also causes a significant improvement in a phenotype, in vivo, associated with type II diabetes and/or obesity (e.g., the chemical agent causes one or more of the following changes: lowers LDL cholesterol, raises HDL cholesterol, lowers body weight, decreases the rate of body weight gain in response to a diet high in fat, decreases insulin resistance, increases glucose tolerance and/or decreases fat pad mass (in rats or mice) in response to a high fat diet).

Animal models can be used to validate the efficacy of a chemical agent that modulates a biological activity of a GPR105 protein in vitro. For example, the effect of a chemical agent on blood pressure can be directly determined using, for example, a radiotelemetry technique (see, e.g., P. A. Mills et al., “A New Method for Measurement of Blood Pressure, Heart Rate, and Activity in the Mouse by Radiotelemetry,” J. Appl. Physiol. 88(5):1537-1544, 2000). Again by way of example, the effect of a chemical agent on blood pressure can be indirectly determined using, for example, a tail-cuff technique (see, e.g., B. N. Van Vliet et al., “Direct and Indirect Methods Used to Study Arterial Blood Pressure,” J. Pharmacol. Toxicol. Methods 44(2):361-373, 2000).

Again by way of example, the effect of a chemical agent on body weight can be determined using an obesity model wherein obesity is induced by a high fat diet or by using an obese mutant mouse model (e.g., ob/ob mice) (see, e.g., M. Tschop and M. L. Heiman, “Rodent Obesity Models: An Overview,” Exp. Clin. Endocrinol. Diabetes, 109(6):307-319, 2001).

By way of further example, the effect of a chemical agent on a component of type II diabetes can be measured using a streptozotocin-induced diabetic model, or, for example, by using a spontaneous mutant model such as the obese Zucker rats (fa/fa rats) or the db/db mice (see, e.g., D. Methe, “Dyslipidemia and Diabetes: Animal Models,” Diabetes Metab. 21(2): 106-111, 1995).

In another aspect, the present invention provides methods for determining the effect of a chemical agent on GPR105 activity. The methods of this aspect of the invention each include the steps of: (a) observing a change in a GPR105-mediated response in a living cell, in vitro, in response to a chemical agent; and (b) confirming that the observed change in the GPR105-mediated response occurs in vivo.

In the practice of this aspect of the invention, a change in a GPR105-mediated response can be observed using, for example, any of the techniques described herein for determining whether a chemical agent modulates the biological activity of a GPR105 protein in a living cell. An observed change in a GPR105-mediated response can be confirmed, in vivo, using any of the methods described herein for validating, in vivo, the modulating effect of a chemical agent on GPR105 biological activity.

In a further aspect, the present invention provides methods for identifying a chemical agent that ameliorates a symptom of type II diabetes or obesity. The methods of this aspect of the invention include the steps of: (a) contacting a living cell, in vitro, with a chemical agent, wherein the living cell expresses a GPR105 protein having a biological activity; (b) determining whether the chemical agent modulates the biological activity of the GPR105 protein in the living cell; and (c) determining, in vivo, whether the chemical agent ameliorates a symptom of type II diabetes or obesity. Examples of symptoms of type II diabetes and/or obesity include increased insulin resistance, increased body mass, and a decreased rate of glucose clearance from the blood stream.

In a further aspect, the present invention provides an isolated nucleic acid molecule (SEQ ID NO:2) that encodes a chimpanzee GPR105 protein (SEQ ID NO:3). The present invention also provides an isolated GPR105 protein (SEQ ID NO:3) that can be obtained, for example, by expressing the cDNA molecule disclosed in SEQ ID NO:2 in living cells. The present invention also provides a further isolated nucleic acid molecule (SEQ ID NO:4) that encodes the GPR105 protein having the amino acid sequence set forth in SEQ ID NO:3.

The following examples merely illustrate the best mode now contemplated for practicing the invention, but should not be construed to limit the invention.

### EXAMPLE 1

This Example shows that an antisense oligonucleotide directed against GPR105 reduced the expression of GPR105 in transgenic mice expressing the antisense oligonucleotide, and reduced the extent of phenotypes associated with Metabolic Syndrome.

Several large mouse crosses were performed, including crosses between C57BL/6 ApoE −/− x C3H/HEJ ApoE −/− crosses. The F2 mice resulting from this cross were analyzed for various genetic as well as phenotype traits relating to metabolic disorders and these traits were correlated with gene expression analysis. Central to the genetic analysis was a likelihood-based test for causality that takes into account genotypic, RNA, and clinical data in a segregating population to identify genes in the trait-specific transcriptional network that are under the control of multiple Quantitative Trait Loci (QTLs) for the trait of interest, but still upstream of the trait. The mouse crosses, and the gene analysis methodology are described by E. E. Schadt et al., Nature Genetics 37(7):710-717, 2005, which publication is incorporated herein by reference.

GPR105 was identified as a candidate gene that may cause adiposity. Several antisense oligonucleotides (ASOs) were evaluated, in vitro, in NIH-3T3 cells, and one oligonucleotide (referred to as the “effective GPR105 antisense oligonucleotide”) caused greater than 90% reduction of GPR105 expression.

The effective GPR105 antisense oligonucleotide was then used to reduce GPR105 expression in vivo in diet induced obesity (DIO) mice. The experiment used a saline control (referred to as the “vehicle”), a scrambled antisense oligonucleotide as a negative control, and an SCD-1 antisense oligonucleotide as a positive control. This experiment was conducted for four weeks, a timeframe in which the positive control demonstrates efficacy. The results are shown in Table 1. The following abbreviations are used in Table 1: GPR105, the effective GPR105 antisense oligonucleotide; SEM, standard error of the mean.

TABLE 1

| | % Weight gain | | | | | | | |
|---|---|---|---|---|---|---|---|---|
| | Vehicle | | GPR105 | | SCD-1 | | Scrambled | |
| Week | Mean | SEM | Mean | SEM | Mean | SEM | Mean | SEM |
| 1 | 8.35 | 1.2 | 5.08 | 1.61 | 12.13 | 1.06 | 11.87 | 1.49 |
| 2 | 18.47 | 2.38 | 10.47 | 1.49 | 18.67 | 2.52 | 22.78 | 2.44 |
| 3 | 27.58 | 3.28 | 15.08 | 2.25 | 23.48 | 3.42 | 34.22 | 3.18 |
| 4 | 37.92 | 1.83 | 18.2 | 2.49 | 25.55 | 3.35 | 38.93 | 2.52 |

As shown in Table 1, the mice treated with the SCD-1 antisense oligonucleotide, and the effective GPR105 antisense oligonucleotide, were significantly resistant to weight gain induced by a high-fat diet. After four weeks of treatment, the mice treated with the effective GPR105 antisense oligonucleotide had less than half the weight of the animals treated with the scrambled oligonucleotide or saline control, with no significant difference in food intake.

In order to confirm that a reduction of GPR105 mRNA was caused by the effective GPR105 antisense oligonucleotide, quantitative PCR analysis was performed on RNA isolated from liver, adipose, and kidney. In animals treated with the effective GPR105 antisense oligonucleotide, greater than 85% reduction of GPR105 MRNA levels was observed in liver and kidney, whereas adipose tissue showed a lesser reduction of 37%. Similar reduced levels of GPR105 mRNA were also observed in animals treated with the SCD-1 antisense oligonucleotide. The results of this experiment are shown in Table 2.

TABLE 2

| | Liver | | Adipose Tissue | | Kidney | |
|---|---|---|---|---|---|---|
| | GPR105 | SCD-1 | GPR105 | SCD-1 | GPR105 | SCD-1 |
| Group | relative exp | | relative exp | | relative exp | |
| Vehicle | 1.00 | 1.00 | 1.00 | 1.00 | 1.00 | 1.00 |
| Vehicle | 1.83 | 0.81 | 0.66 | 1.60 | 0.81 | 0.49 |
| Vehicle | 0.53 | 0.33 | 0.96 | 1.39 | 0.55 | 0.72 |
| Vehicle | 0.68 | 1.21 | 0.67 | 0.70 | 0.48 | 1.05 |
| Vehicle | 0.43 | 0.60 | 0.89 | 0.52 | 0.46 | |
| Vehicle (Mean ± SEM) | 0.89 ± 0.25 | 0.79 ± 0.15 | 0.84 ± 0.07 | 1.04 ± 0.2 | 0.66 ± 0.11 | 0.82 ± 0.13 |
| Antisense | 0.02 | 0.12 | 0.65 | 0.86 | 0.10 | 0.35 |
| Antisense | 0.01 | 0.15 | 0.40 | 0.82 | 0.09 | 0.89 |
| Antisense | 0.01 | 0.19 | 0.44 | 0.83 | 0.11 | 0.48 |
| Antisense | 0.27 | 0.10 | 0.69 | 0.30 | 0.09 | 0.23 |
| Antisense | 0.07 | 0.09 | 0.49 | 0.27 | 0.07 | 0.30 |
| Antisense (Mean ± SEM) | 0.08 ± 0.05 | 0.13 ± 0.02 | 0.53 ± 0.06 | 0.62 ± 0.14 | 0.09 ± 0.01 | 0.45 ± 0.12 |
| Scrambled Oligo | 0.96 | 0.37 | 1.22 | 1.08 | 0.45 | 0.47 |
| Scrambled Oligo | 1.09 | 1.43 | 1.19 | 1.66 | 1.01 | 0.96 |
| Scrambled Oligo | 0.63 | 0.44 | 1.38 | 0.98 | 0.57 | 0.62 |
| Scrambled Oligo | 0.89 | 0.50 | 1.49 | 0.98 | 0.46 | 0.49 |
| Scrambled Oligo | 0.57 | 0.54 | 1.60 | 0.88 | 0.35 | |
| Scrambled Oligo (Mean ± SEM) | 0.83 ± 0.1 | 0.66 ± 0.2 | 1.38 ± 0.08 | 1.12 ± 0.14 | 0.57 ± 0.12 | 0.63 ± 0.11 |

Since significant resistance to weight gain was achieved with the effective GPR105 antisense oligonucleotide, insulin levels were also measured. In this experiment, insulin levels were measured from mouse plasma at the end of the 4 week antisense oligonucleotide experiment described in connection with Table 1. Samples were analyzed in duplicate using an ELISA assay (the ELISA kit was purchased from CrystalChem, IL, USA, Catalog No. 90060) and the corresponding mean, and the mean of the group of animals, is reported.

Significant increases in insulin levels were observed in mice treated with the vehicle and scrambled antisense oligonucleotides as compared to mice treated with the effective GPR105 antisense oligonucleotide and the SCD-1 antisense oligonucleotide (p<0.005 and 0.01, respectively ) for similar glucose levels. The results of this experiment are shown in Table 3.

TABLE 3
Insulin Measurement ng/ml

| | Mouse | Dupl | Dupl | | Group | |
|---|---|---|---|---|---|---|
| Treatment | # | #1 | #2 | Mean | Mean | SEM |
| Vehicle | 1 | 2.9 | 3.2 | 3.0 | 3.4 | 0.6 |
| Vehicle | 2 | 2.4 | 2.5 | 2.4 | | |
| Vehicle | 3 | 3.1 | 3.0 | 3.1 | | |
| Vehicle | 4 | 2.8 | 2.5 | 2.6 | | |
| Vehicle | 5 | 5.7 | 5.6 | 5.6 | | |
| GPR105ASO | 6 | 0.7 | 0.8 | 0.7 | 1.1 | 0.2 |
| GPR105ASO | 7 | 1.0 | 0.7 | 0.8 | | |
| GPR105ASO | 8 | 1.3 | 1.2 | 1.3 | | |
| GPR105ASO | 9 | 1.8 | 1.8 | 1.8 | | |
| GPR105ASO | 10 | 0.7 | 0.7 | 0.7 | | |
| SCD-1ASO | 11 | 0.8 | 0.7 | 0.8 | | |
| SCD-1ASO | 12 | 1.8 | 1.6 | 1.7 | 1.5 | 0.2 |
| SCD-1ASO | 13 | 2.0 | 1.7 | 1.8 | | |
| SCD-1ASO | 14 | 1.3 | 1.2 | 1.2 | | |
| SCD-1ASO | 15 | 2.1 | 1.9 | 2.0 | | |
| Scrambled ASO | 16 | 1.4 | 1.3 | 1.4 | 2.4 | 0.3 |
| Scrambled ASO | 17 | 1.9 | 2.2 | 2.1 | | |
| Scrambled ASO | 18 | 2.3 | 2.8 | 2.6 | | |
| Scrambled ASO | 19 | 3.5 | 3.3 | 3.4 | | |
| Scrambled ASO | 20 | 3.0 | 2.5 | 2.8 | | |

The data shown in Table 3 suggest that the obese mice were becoming insulin resistant in relation to the mice treated with the effective GPR105 antisense oligonucleotide or the SCD-1 antisense oligonucleotide.

White adipose tissue (WAT) was isolated (and weighed) from the mice treated with the effective GPR105 antisense oligonucleotide, the vehicle, and the scrambled antisense oligonucleotide negative control. The weight of the epididymal fat pads is shown in Table 4.

TABLE 4

| | Weight (g) | Mean | SEM |
|---|---|---|---|
| Vehicle | 0.26 | 0.44 | 0.16 |
| Vehicle | 0.57 | | |
| Vehicle | 1.02 | | |
| GPR105 ASO | 0.17 | 0.16 | 0.02 |
| GPR105 ASO | 0.17 | | |
| GPR105 ASO | 0.11 | | |
| GPR105 ASO | 0.17 | | |
| SCD-1 ASO | 0.37 | 0.29 | 0.04 |
| SCD-1 ASO | 0.39 | | |
| SCD-1 ASO | 0.29 | | |
| SCD-1 ASO | 0.19 | | |
| SCD-1 ASO | 0.23 | | |
| Scrambled ASO | 0.15 | 0.46 | 0.17 |
| Scrambled ASO | 0.15 | | |
| Scrambled ASO | 0.25 | | |
| Scrambled ASO | 0.95 | | |
| Scrambled ASO | 0.81 | | |

The weight of the epididymal fat pads in the animals treated with the effective GPR105 antisense oligonucleotide were significantly reduced compared to the vehicle and scrambled antisense oligonucleotide controls (p<0.05). This data demonstrates that a significant amount of the weight loss observed in the DIO study was due to decreases in fat pad weight.

Since the adipose tissue weight was significantly decreased in the mice treated with the effective GPR105 antisense oligonucleotide, adipose tissues was also sectioned for histological analysis. WAT sections from both inguinal and epididymal regions were obtained. The sections were 5μm thick and they were fixed and stained using heamatoxylin and Eosin. The researcher was blinded to the tissue samples and a microscope eye piece with plain cross-hairs was used. A 20× objective was used and a field was chosen where both the horizontal and vertical lines were overlaying the cross section. Each time one of the cross hairs intersected the adipocyte wall provides a quantitative measure of adipocyte size. This was repeated for 10 different regions throughout the histology section. The value that was obtained is an Lm measurement, where Lm=nL/I, n equals the number of lines superimposed, L is the length of the line and I is the total number of intercepts (W. M. Thurlbeck, “Measurement of Pulmonary Emphysema,” American Review of Respiratory Disease 95:752-764, 1967). Therefore, the higher the value of Lm the larger the adipocyte. The results of this analysis for inguinal and epididymal WAT are depicted below in Table 5.

TABLE 5

| | Inguinal WAT | | | Epididymal WAT | | |
|---|---|---|---|---|---|---|
| | Lm (μM) | | | | | |
| Group | n | Mean | SEM | n | Mean | SEM |
| Vehicle | 4 | 91.0 | 9.7 | 3 | 109.5 | 13.7 |
| GPR105 ASO | 3 | 44.6 | 5.5 | 4 | 68.2 | 7.4 |
| SCD-1 ASO | 5 | 73.4 | 7 | 5 | 103.7 | 10 |
| Scrambled ASO | 5 | 73.2 | 7 | 5 | 91.8 | 8.9 |

As shown in Table 5, the mice treated with the effective GPR105 antisense oligonucleotide had both a significant decrease in adipose tissue weight and size. In fact, the size of the adipocytes in the inguinal WAT from the mice treated with the effective GPR105 antisense oligonucleotide was only 60% (p<0.01) of either the scrambled ASO or vehicle control animals. In the epididymal WAT the adipocyte size of the mice treated with the effective GPR105 antisense oligonucleotide was 74% of the size of the scrambled ASO or vehicle control animals (difference with vehicle p<0.05, difference with scrambled, p=0.063). These data clearly demonstrate a substantial effect on adipose cell size in mice placed on a high fat/high carbohydrate diet when treated with the effective GPR105 antisense oligonucleotide.

### EXAMPLE 2

This Example describes the development of a cell-based functional assay to identify modulators (e.g., agonists or antagonists) of GPR105 activity.

A region of the chimpanzee genome was identified which was predicted to encode a simian GPR105 protein. The human P2RY14 protein sequence (Genbank RefSeq NP_{—}055694.2) was used to perform a TBLASTN search against the Ensembl chimpanzee (Pan troglodytes) Ab-initio/SNAP DNA database using the default configuration parameters for TBLASTN, with the exception that the query sequence was not filtered to remove regions of low complexity.

A near-perfect match (99.1 percent identity) corresponding to the complete 338 amino acid residues of the human P2RY14 protein sequence was identified as GENSCAN00000068233. The nucleic acid sequence of the 1017 bp predicted cDNA is shown in SEQ ID NO:2, and encodes the predicted GPR105 protein having the amino acid sequence shown in SEQ ID NO:3. The 1017 bp predicted cDNA (SEQ ID NO:2) is located on chimpanzee chromosome 2, with genomic coordinates 155619963 to 155620976 on the negative strand.

The chimpanzee GPR105 sequence (SEQ ID NO:2) was chemically synthesized (BIO S & T, Montreal, PQ, Canada), modified by the addition of restriction sites at the 5′ and 3′ ends to produce the cDNA having the sequence shown in SEQ ID NO:4, and cloned into the pcDEF3 expression vector (Goldman et al., “Modifications of Vectors pEF-BOS, pcDNA1 and pcDNA3 Result in Improved Convenience and Expression,” Biotechniques 21:1013-1015, 1996). A clonal cell line was selected using limited dilution cloning and testing for Ca++ release in HEK cells co-expressing simial GPR105 and Gqi5 by FLIPR, as previously described (K. Freeman et al., Genomics 78:124-128, 2001). This cell-based functional assay can be utilized to identify modulators (e.g., agonists or antagonists) of GPR105 activity.

While illustrative embodiments have been illustrated and described, it will be appreciated that various changes can be made therein without departing from the spirit and scope of the invention.

1. A method for determining whether a chemical agent modulates the biological activity of a GPR105 protein, the method comprising the steps of:
(a) contacting a living cell, in vitro, with a chemical agent, wherein the living cell expresses a GPR105 protein having a biological activity;
(b) determining whether the chemical agent modulates the biological activity of the GPR105 protein in the living cell; and
(c) validating, in vivo, the modulating effect of the chemical agent on the biological activity of the GPR105 protein.

2. A method of claim 1, wherein the chemical agent consists essentially of a chemical compound.

3. A method of claim 1, wherein the chemical agent consists essentially of a protein.

4. A method of claim 1, comprising the step of determining whether the agent decreases the biological activity of the GPR105 protein.

5. A method of claim 1, comprising the step of determining whether the agent increases the biological activity of the GPR105 protein.

6. A method of claim 1, wherein the cell is selected from the group consisting of a CHO cell and an HTK cell.

7. A method of claim 1, wherein the GPR105 protein is at least 70% identical to a GPR105 protein consisting of the amino acid sequence set forth in SEQ ID NO:1.

8. A method of claim 1, wherein the GPR105 protein is at least 80% identical to a GPR105 protein consisting of the amino acid sequence set forth in SEQ ID NO:1.

9. A method of claim 1, wherein the GPR105 protein is at least 90% identical to a GPR105 protein consisting of the amino acid sequence set forth in SEQ ID NO:1.

10. A method of claim 1, wherein the GPR105 protein is at least 95% identical to a GPR105 protein consisting of the amino acid sequence set forth in SEQ ID NO:1.

11. A method of claim 1, wherein the GPR105 protein is at least 99% identical to a GPR105 protein consisting of the amino acid sequence set forth in SEQ ID NO:1.

12. A method of claim 1, wherein the modulating effect of the chemical agent on the GPR105 is validated by measuring the effect of the chemical agent on a physiological response selected from the group consisting of the concentration of LDL cholesterol in blood plasma, the concentration of HDL cholesterol in blood plasma, body weight, the rate of body weight gain in response to a high fat diet, insulin resistance, and glucose clearance from the blood stream.

13. A method of claim 1, wherein a population of living cells are contacted, in vitro, with a chemical agent, wherein all, or substantially all, of the living cells express a GPR105 protein having a biological activity.

14. An automated method of claim 1.

15. A method for determining the effect of a chemical agent on GPR105 activity in a living cell, the method comprising the steps of:
(a) observing a change in a GPR105-mediated response in a living cell, in vitro, in response to a chemical agent; and
(b) confirming that the observed change in the GPR105-mediated response occurs in vivo.

16. A method of claim 15, wherein the GPR105-mediated response is selected from the group consisting of the concentration of LDL cholesterol in blood plasma, the concentration of HDL cholesterol in blood plasma, body weight, the rate of body weight gain in response to a high fat diet, insulin resistance, and glucose clearance from the blood stream.

17. A method of claim 15, wherein the chemical agent consists essentially of a chemical compound.

18. A method of claim 1, wherein the chemical agent consists essentially of a protein.

19. A method of claim 15, wherein the change in the GPR105-mediated response is an increase in the GPR105-mediated response.

20. A method of claim 15, wherein the change in the GPR105-mediated response is a decrease in the GPR105-mediated response.

21. A method for identifying a chemical agent that ameliorates a symptom of type II diabetes or obesity, the method comprising the steps of:
(a) contacting a living cell, in vitro, with a chemical agent, wherein the living cell expresses a GPR105 protein having a biological activity;
(b) determining whether the chemical agent modulates the biological activity of the GPR105 protein in the living cell; and
(c) determining, in vivo, whether the chemical agent ameliorates a symptom of type II diabetes or obesity.

22. A method of claim 21, wherein the symptom of type II diabetes or obesity is selected from the group consisting of insulin resistance, body mass, and glucose clearance from the blood stream.

23. An isolated nucleic acid molecule that encodes a GPR105 protein, wherein the isolated nucleic acid molecule comprises the nucleic acid sequence set forth in SEQ ID NO:2.

24. An isolated nucleic acid molecule of claim 23, consisting of the nucleic acid sequence set forth in SEQ ID NO:2.

25. An isolated GPR105 protein comprising the amino acid sequence set forth in SEQ ID NO:3.

26. An isolated GPR105 protein of claim 25, consisting of the amino acid sequence set forth in SEQ ID NO:3.


SEQUENCE LISTING (5 sequences)

SEQ ID NO: 1 - protein, 338 aa, Homo sapiens
MINSTSTQPPDESCSQNLLITQQIIPVLYCMVFIAGILLNGVSGWIFFYVPSSESFIIYLKNIVIADFVMSLTFPFKILGDSGLGPWQLNVFVCRVSAVLFYVNMYVSIVFFGLISFDRYYKIVKPLWTSFIQSVSYSKLLSVIVWMLMLLLAVPNIILTNQSVREVTQIKCIELKSELGRKWHKASNYIFVAIFWIVFLLLIVFYTAITKKIFKSHLKSSRNSTSVKKKSSRNIFSIVFVFFVCFVPYHIARIPYTKSQTEAHYSCQSKEILRYMKEFTLLLSAANVCLDPIIYFFLCQPFREILCKKLHIPLKAQNDLDISRIKRGNTTLESTDTL

SEQ ID NO: 2 - DNA, 1017 bp, Chimpanzee
  CDS (1)..(1017)
atgatcaattcaacctccacacagcctccagatgaatcctgctctcagaacctcctgatcactcagcagatcattcctgtgctgtactgtgtggtcttcattgcgggaatcctcctcaatggagtgtcaggatggatattcttttacgtgcccagctctaagagtttcattgtctatctcaagaacattgttattgctgactttgtgatgagcctgacttttcctttcaagatccttggtgactcaggccttggtccctggcagctgaacgtgtttgtgtgcagggtctctgccgtgctcttctacgtcaacatgtacgtcagcattgtgttctttgggctcatcagctttgatagatattataaaattgtaaagcctctttggacttctttcatccagtcagtgagttacagcaaactcctgtcagtgatagtatggatgctcatgctgctccttgctgttccaaatattattctcaccaaccagagtgttagggaggttacacaaataaaatgtatagaactgaaaagtgaactgggacggaagtggcacaaagcatcaaactacatcttcgtggccatcttctggattgtgtttcttttgttaatcgttttctatactgctatcacaaagaaaatctttaagtcccaccttaagtcaagtcggaattccacttcggtcaaaaagaaatctagccgcaacatattcagcattgtgttcgtgttttttatctgttttgtaccttaccatattgccagaatcccctacacaaagagtcagaccgaagctcattacagctgccagtcaaaagaaatcttgcggtatatgaaagaattcactctgctactatctgctgcaaatgtatgcttggaccctattatttatttctttctatgccagccatttagggaaatcttatgtaagaaattgcacattccattaaaagctcagaatgacctagacatttccagaatcaaaagaggaaatacaacacttgaaagcacagatactttgtga

SEQ ID NO: 3 - protein, 338 aa, Chimpanzee
MINSTSTQPPDESCSQNLLITQQIIPVLYCVVFIAGILLNGVSGWIFFYVPSSKSFIVYLKNIVIADFVMSLTFPFKILGDSGLGPWQLNVFVCRVSAVLFYVNMYVSIVFFGLISFDRYYKIVKPLWTSFIQSVSYSKLLSVIVWMLMLLLAVPNIILTNQSVREVTQIKCIELKSELGRKWHKASNYIFVAIFWIVFLLLIVFYTAITKKIFKSHLKSSRNSTSVKKKSSRNIFSIVFVFFICFVPYHIARIPYTKSQTEAHYSCQSKEILRYMKEFTLLLSAANVCLDPIIYFFLCQPFREILCKKLHIPLKAQNDLDISRIKRGNTTLESTDTL

SEQ ID NO: 4 - DNA, 1037 bp, Chimpanzee
  CDS (10)..(1029)
ggtaccaccaccatgatcaattcaacctccacacagcctccagatgaatcctgctctcagaacctcctgatcactcagcagatcattcctgtgctgtactgtgtggtcttcattgcgggaatcctcctcaatggagtgtcaggatggatattcttttacgtgcccagctctaagagtttcattgtctatctcaagaacattgttattgctgactttgtgatgagcctgacttttcctttcaagatccttggtgactcaggccttggtccctggcagctgaacgtgtttgtgtgcagggtctctgccgtgctcttctacgtcaacatgtacgtcagcattgtgttctttgggctcatcagctttgatagatattataaaattgtaaagcctctttggacttctttcatccagtcagtgagttacagcaaactcctgtcagtgatagtatggatgctcatgctgctccttgctgttccaaatattattctcaccaaccagagtgttagggaggttacacaaataaaatgtatagaactgaaaagtgaactgggacggaagtggcacaaagcatcaaactacatcttcgtggccatcttctggattgtgtttcttttgttaatcgttttctatactgctatcacaaagaaaatctttaagtcccaccttaagtcaagtcggaattccacttcggtcaaaaagaaatctagccgcaacatattcagcattgtgttcgtgttttttatctgttttgtaccttaccatattgccagaatcccctacacaaagagtcagaccgaagctcattacagctgccagtcaaaagaaatcttgcggtatatgaaagaattcactctgctactatctgctgcaaatgtatgcttggaccctattatttatttctttctatgccagccatttagggaaatcttatgtaagaaattgcacattccattaaaagctcagaatgacctagacatttccagaatcaaaagaggaaatacaacacttgaaagcacagatactttgtgagcggccgc

SEQ ID NO: 5 - protein, 339 aa, Chimpanzee
TMINSTSTQPPDESCSQNLLITQQIIPVLYCVVFIAGILLNGVSGWIFFYVPSSKSFIVYLKNIVIADFVMSLTFPFKILGDSGLGPWQLNVFVCRVSAVLFYVNMYVSIVFFGLISFDRYYKIVKPLWTSFIQSVSYSKLLSVIVWMLMLLLAVPNIILTNQSVREVTQIKCIELKSELGRKWHKASNYIFVAIFWIVFLLLIVFYTAITKKIFKSHLKSSRNSTSVKKKSSRNIFSIVFVFFICFVPYHIARIPYTKSQTEAHYSCQSKEILRYMKEFTLLLSAANVCLDPIIYFFLCQPFREILCKKLHIPLKAQNDLDISRIKRGNTTLESTDTL